(19) United States
(12) Patent Application Publication
(10) Pub. No.: US 2021/0267981 A1
(43) Pub. Date: Sep. 2, 2021

(54) COMPOUNDS FOR THE TREATMENT OF HIV INFECTION AND OTHER DISEASES CAUSED BY RNA VIRUSES

(71) Applicant: UNIVERSIDAD CATOLICA DE VALENCIA "SAN VICENTE MARTIR", Valencia (ES)

(72) Inventors: Jose GALLEGO SALA, La Canada, Valencia (ES); Silvia PRADO MARTIN, Valencia (ES); Jose ALCAMI PERTEJO, Madrid (ES)

(21) Appl. No.: 17/256,087

(22) PCT Filed: Jun. 24, 2019
(86) PCT No.: PCT/ES2019/070440

§ 371 Date: Dec. 24, 2020

(30) Foreign Application Priority Data
Jun. 25, 2018 (ES) P201830637

(51) Int. Cl. A61K 31/519; A61K 31/138; A61K 31/497

(52) U.S. Cl. CPC A61K 31/519; A61K 31/497; A61K 31/138

(57) ABSTRACT

Use of organic compounds for the treatment of the infection caused by human immunodeficiency virus type 1 (HIV-1), as well as other diseases caused by RNA viruses is disclosed.

### FIELD OF THE INVENTION

The present invention relates to the use of organic compounds for the treatment of the infection caused by human immunodeficiency virus type 1 (HIV-1), as well as other diseases caused by RNA viruses.

### BACKGROUND OF THE INVENTION

According to the World Health Organization, at the end of 2016, there were more than 36 million people infected with HIV worldwide, with 1.8 million new infections and 1 million related deaths from AIDS-related diseases being recorded in the same year.

In recent years, antiretroviral therapy has lowered the incidence and mortality rates of the disease. Treatment is based on combinations of three or four drugs which inhibit viral protease, reverse transcriptase, or integrase, or block its cell entry. However, this therapy does not eliminate the infection. The onset of resistances and the lack of an effective vaccine further support the need to identify new drugs acting on alternative targets of the virus. Therefore, the development of new therapies which successfully eliminate the infection caused by HIV is still greatly needed today.

RNA plays a central role in the functioning of living beings, and many human, bacterial, and viral RNA molecules exhibit considerable therapeutic potential that is yet to be utilized. Today, two strategies are used to address receptors of this type. The first strategy is based on the generation of antisense agents or iRNA designed for pairing with the target RNA and thereby promoting its degradation or blocking its translation. The second strategy consists of the synthesis of small organic molecules designed for specifically recognizing the cavities formed by tertiary RNA structures and thus interfering with their function.

Functional and structured RNA motifs are not readily accessible to antisense agents and have the advantage of exhibiting a significant sequence and/or three-dimensional structure conservation. This factor is important for the development of anti-infective agents, as it could result in a slower onset of resistances to drugs acting on these structures. However, with the exception of several antibiotics which interact with bacterial ribosomal RNA sites, the development of new RNA-targeting drugs has been hampered by the difficulties imposed by these structures which have a limited physicochemical diversity and are often flexible. For this strategy to succeed, new chemical backbones and new mechanisms for the specific recognition of structured RNA must be identified.

From the pharmacological viewpoint, processes contributing to the biogenesis and packaging of the RNA molecules of the human immunodeficiency virus type 1 (HIV-1) are clearly not utilized. Activities such as transcription, splicing, nuclear export, and packaging involve viral RNA domains and virus-encoded proteins with the potential for providing selectivity in relation to host components, but they do not constitute the target of any of the antiretroviral drugs available on the market today. These agents block the first steps of the viral cycle, including the entry, reverse transcription, and integration of the virus, as well as the generation of ineffective virions through protease inhibition, but they do not disrupt most of the activities following integration of the virus. The discovery and development of a drug targeting one or more steps of HIV-1 RNA biogenesis may provide new therapeutic opportunities to cure the disease and may also help to mitigate the current problem of resistance to antiretroviral treatments.

Rev, a virus-encoded, 116-amino acid protein which adopts a helix-loop-helix shape, binds to the Rev response element (RRE), a 350-nucleotide structure present in semi-processed and unprocessed viral RNA transcripts. The formation of the RRE-Rev ribonucleoprotein involves the association of several Rev monomers with RRE through a cooperative process involving RNA-protein and protein-protein interactions. This process is triggered by means of a high-affinity interaction between an internal loop located within RRE subdomain IIB and the RNA-binding α-helix (referred to hereinafter as Rev₃₄₋₅₀) of the first Rev monomer. Once formed, the RRE-Rev complex binds to cellular factor Crm1 to induce the nuclear export of semi-processed and unprocessed viral RNA molecules, an essential step in the late phase of the viral cycle. Rev-mediated nuclear export of RNA represents a potential target for anti HIV-1 therapy.

In this sense, small molecules acting on RRE-Rev interaction have been found. For example, Rev₃₄₋₅₀-mimicking small molecules with antiretroviral activity are described (Gonzalez-Bulnes et al., Angewandte Chemie-International Edition 52(50) (2013) 13405-13409). It has also been described that clomifene affects the formation of RRE-Rev complex in vitro and exhibits antiretroviral activity ex vivo, by means of blocking the transcription and inhibiting the function of Rev (S. Prado et al., Biochem Pharmacol 107 (2016) 14-28).

Nevertheless, there is a need to develop new compounds for treating diseases caused by RNA viruses, particularly by the HIV-1 virus, and especially compounds acting in stages following the integration of the virus.

### SUMMARY OF THE INVENTION

The researchers have found that compounds of formula (I), (II), and (III) are capable of inhibiting RRE-Rev interaction, both the interaction of subdomain IIB of the RRE RNA with the RNA-binding α-helix of Rev and the entire RRE-Rev interaction. These compounds also exhibit antiretroviral activity.

Therefore, a first aspect of the invention relates to a compound of formula (I) as described herein, or a salt or solvate thereof, for use in the prevention or treatment of infections caused by RNA viruses.

In a second aspect, the invention relates to a compound of formula (II) as described herein, or a salt or solvate thereof, for use in the prevention or treatment of infections caused by RNA viruses.

In a third aspect, the invention relates to a compound of formula (III) as described herein, or a salt or solvate thereof, for use in the prevention or treatment of infections caused by RNA viruses.

Another aspect of the invention relates to the use of a compound of formula (I), (II), or (III) as described herein, or a salt or solvate thereof, in the preparation of a medicinal product for the prevention or treatment of infections caused by RNA viruses.

Another aspect of the invention relates to a method of preventing or treating infections caused by RNA viruses which comprises administering a therapeutically effective amount of a compound of formula (I), (II), or (III) as described herein, or a salt or solvate thereof.

In an additional aspect, the invention relates to a pharmaceutical composition comprising a compound of formula (I), (II), or (III) as described herein, or a salt or solvate thereof, for use in the prevention or treatment of infections caused by RNA viruses.

### DESCRIPTION OF THE FIGURES

FIG. 1. Chemical structure of compounds 1a, 2a, and 2b.

FIG. 2. Inhibition of IIBh-Rev₃₄₋₅₀ interaction and of the formation of the complete RRE-Rev complex by compounds 1a, 2a, and 2b. (a) Inhibition curves of IIBh-Rev₃₄₋₅₀ obtained with fluorescence anisotropy experiments. (b) Inhibition of the formation of complete RRE-Rev complex analyzed using EMSA. In (a), the error bars represent the standard deviation of three independent experiments.

FIG. 3. Recognition of the IIBh RNA fork by compounds 1a, 2a, and 2b. IIBh-binding curves obtained with fluorescence intensity experiments in the absence (black circles) and presence of a 100-fold molar excess of unlabeled competing RNA (RNAtCys; grey triangles) or unlabeled competing double-helix DNA (LTRd; grey inverted triangles). The error bars represent the standard deviations of two independent experiments.

FIG. 4. Cellular assays for compounds 1a, 2a, and 2b. (a) Antiviral activity as a function of the concentration of the compound in HIV-1 cell infection experiments. Cell toxicity (black squares) is also shown in the same graphs. (b) Inhibition of the steps following the integration of HIV-1: the cells were transfected with a DNA plasmid encoding a full-length virus containing a luciferase reporter gene. In all the cases, the results are expressed as percentage of luminescence (RLU), where 100% is the level of viral replication obtained in the presence of the vehicle used for dissolving the compounds.

FIG. 5. Effect of compound 1a on HIV-1 RNA processing. The processed, semi-processed, and completely processed HIV-1 RNA transcripts were quantified by means of RT-qPCR after the isolation of RNA from cells transfected with an HIV-1 pNL4.3 plasmid and treated with the inhibitor. The results are expressed as a relative amount of RNA (RQ), using untreated cells as a reference (RQ=1).

### DETAILED DESCRIPTION OF THE INVENTION

The term “halogen” refers to bromine, chlorine, iodine, or fluorine.

The term “alkyl” refers to a linear or branched alkane derivative which contains from 1 to 6 (“C₁-C₆ alkyl”), preferably from 1 to 3 (“C₁-C₃ alkyl”), carbon atoms and is attached to the rest of the molecule through a single bond. Illustrative examples of alkyl groups include methyl, ethyl, n-propyl, i-propyl, n-butyl, i-butyl, t-butyl, pentyl, hexyl.

The term “cycloalkyl” refers to a cycloalkane derivative containing 3 to 7 (“C₃-C₇ cycloalkyl”), preferably 3 to 6 (“C₃-C₆ cycloalkyl”), carbon atoms. Illustrative examples of cycloalkyl groups include cyclopropyl, cyclobutyl, cyclopentyl, cyclohexyl, etc.

The term “haloalkyl” refers to an alkyl group as defined above, wherein at least one of the hydrogen atoms has been substituted by a halogen group, for example CF₃, CCl₃, CHF₂, CF₂CF₃, etc.

The term “aryl” refers to an aromatic group having between 6 and 12, preferably between 6 and 10, carbon atoms, comprising 1 or 2 aromatic nuclei condensed to one another. Illustrative examples of aryl groups include phenyl, naphthyl, indenyl, phenanthryl, etc.

The term “arylalkyl” refers to an alkyl group as defined above substituted with an aryl group as defined above, such as (C₆-C₁₂)aryl(C₁-C₆)alkyl, (C₆-C₁₀)aryl(C₁-C₆)alkyl, and (C₆-C₁₀)aryl(C₁-C₃)alkyl. Examples of these groups include benzyl, phenylethyl, phenylpropyl, naphthylmethyl, etc.

The term “heterocyclyl” refers to a monocyclic, bicyclic, or tricyclic system which can be partially or completely saturated or aromatic (“heteroaryl”) containing 5 to 15, preferably 5 to 10, more preferably 5 or 6, ring atoms containing one or more, specifically one, two, three, or four ring heteroatoms independently selected from N, O, and S, and the remaining ring atoms being carbon.

The aforementioned groups can be optionally substituted in one or more available positions with one or more suitable groups such as OR, SR, SOR, SO₂R, OSO₂R, SO₃R, NO₂, N(R)₂, N(R)COR, N(R)SO₂R, CN, halogen, COR, CO₂R, OCOR, OCO₂R, OCON(R)₂, CON(R)₂, C₁-C₆ alkyl, C₂-C₆ alkenyl, C₂-C₆ alkynyl, C₆-C₁₄ aryl, and 5- to 10-membered heterocyclyl, wherein each of the R groups is independently selected from the group consisting of hydrogen, C₁-C₆ alkyl, C₂-C₆ alkenyl, C₂-C₆ alkynyl, C₆-C₁₄ aryl, and 5- to 10-membered heterocyclyl.

The invention also provides “salts” of the compounds described in the present description. By way of illustration, said salts can be acid addition salts, base addition salts, or metal salts, and can be synthesized from the original compounds containing a basic or acidic residue by means of conventional chemical methods known in the art. Such salts are generally prepared, for example, by reacting the free acid or base forms of said compounds with a stoichiometric amount of the suitable base or acid in water or in an organic solvent or in a mixture of both. Illustrative examples of said acid addition salts include inorganic acid addition salts such as, for example, hydrochloride, hydrobromide, hydriodide, sulfate, perchlorate, nitrate, phosphate, etc., organic acid addition salts such as, for example, acetate, maleate, fumarate, citrate, oxalate, succinate, tartrate, malate, mandelate, methanesulfonate, p-toluenesulfonate, camphorsulfonate, etc. Illustrative examples of base addition salts include inorganic base salts such as, for example, ammonium salts, and organic base salts such as, for example, ethylenediamine, ethanolamine, N,N-dialkylenethanolamine, triethanolamine, glutamine, basic amino acid salts, etc. Illustrative examples of metal salts include, for example, sodium, potassium, calcium, magnesium, aluminum, and lithium salts. In a particular embodiment, the salt is an acid addition salt.

Likewise, the compounds described in the present description can be obtained both as free compounds and as solvates (for example, hydrates, alcoholates, etc.), both forms being included within the scope of the present invention. Solvation methods are generally known in the state of the art.

As it is used herein, the expression “pharmaceutical composition” refers to a formulation which has been adapted for administering a predetermined dose of one or more useful therapeutic agents to a cell, a group of cells, an organ, a tissue, or an organism.

The compounds of the invention are administered in a therapeutically effective amount. A “therapeutically effective amount” is understood to be an amount which can provide a therapeutic effect and can be determined by one skilled in the art with commonly used means. The effective amount will vary with the particular disorder that is being treated, the age and the physical condition of the subject that is being treated, the intensity of the disorder, the duration of treatment, the nature of the simultaneous or combination therapy (if any), the specific route of administration, and similar factors within the knowledge and experience of the health professional. Those skilled in the art will appreciate that dosages can also be determined with the guidelines of Goodman and Goldman's The Pharmacological Basis of Therapeutics, 9^{th} edition (1996), appendix II, pages 1707-1711, and Goodman and Goldman's The Pharmacological Basis of Therapeutics, 10th edition (2001), appendix II, pages 475-493.

The pharmaceutical compositions of the invention can include at least one pharmaceutically acceptable vehicle. As it is used herein, the term “pharmaceutically acceptable vehicle” means an inert and non-toxic filler, diluent, encapsulation material, or adjuvant for solid, semisolid, or liquid formulation, of any type which is acceptable for the patient from a pharmacological/toxicological viewpoint and for the manufacturing pharmaceutical chemist from a physical/chemical viewpoint in terms of the composition, formulation, stability, patient acceptance, and bioavailability. Remington's Pharmaceutical Sciences, edition by Gennaro, Mack Publishing, Easton, Pa., 1995 describes various vehicles used in the formulation of pharmaceutical compositions and techniques known for preparing same. The pharmaceutical compositions of the invention include any solid composition (tablets, pills, capsules, pellet, etc.), semi-solid composition (creams, ointments, etc.), or liquid composition (solution, suspension, or emulsion).

The compounds and pharmaceutical compositions of this invention can be administered to a patient using any means known in the art including oral and parenteral routes.

As they are used herein, the terms “treat” and “treatment” include, but are not limited to, reducing, suppressing, inhibiting, alleviating, or affecting the progression, severity, and/or extent of a condition, the possibility of a disease re-emerging or returning after remission. In one embodiment, “treat” can include directly affecting or curing, suppressing, inhibiting, reducing the intensity of, delaying the onset of, reducing the symptoms associated with an infection, or a combination thereof. In another embodiment, “treat” includes delaying progression, speeding up remission, inducing remission, increasing remission, speeding up recovery, increasing the efficacy of or reducing the resistance to alternative treatments, or a combination thereof.

As they are used herein, the terms “prevent” and “prevention” include, but are not limited to, delaying the onset of the symptoms, preventing viral infection, preventing disease relapse, reducing the number or the frequency of relapse episodes, increasing the latency between symptomatic episodes, or a combination thereof.

### Compounds of Formula (I)

In a first aspect, the invention relates to a compound of formula (I)

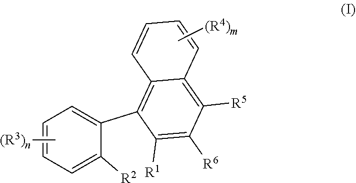

or a salt or solvate thereof, wherein
R¹ and R² are independently selected from H, halogen, OR′, OC(O)R′, C(O)R′, C(O)OR′, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl, optionally substituted, wherein R′ is independently selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl; or
R¹ and R² form, together with the carbon atom to which they are attached, a 5-membered carbocycle or heterocycle;
each R³ and R⁴ is independently selected from halogen, OC(O)R′, SOR′, SO₂R′, C(O)R′, C(O)OR′, CN, NO₂, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl, optionally substituted, and a group of formula (A), wherein R′ is independently selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl; or two contiguous R³ or R⁴ groups form a benzene ring;
R⁵ is selected from H, halogen, OC(O)R′, SOR′, SO₂R′, C(O)R′, C(O)OR′, CN, NO₂, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl, optionally substituted, and a group of formula (A), wherein R′ is independently selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl;
R⁶ is selected from H, halogen, OC(O)R′, SOR′, SO₂R′, C(O)R′, C(O)OR′, CN, NO₂, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl, optionally substituted, and a group of formula (A), wherein R′ is independently selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl;
n represents 0, 1, 2, 3, or 4; and
m represents 0, 1, 2, 3, or 4;
   wherein at least one of R³, R⁴, R⁵, and R⁶ is a group of formula (A)

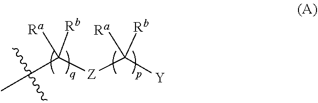

wherein
Z is selected from 5- to 10-membered heterocyclyl, O, S, and NR′, wherein R′ represents H, C₁₋₆ alkyl, C₃₋₇ cycloalkyl, C₁₋₆ haloalkyl, C₆₋₁₂ aryl, (C₆₋₁₂)aryl(C₁₋₆)alkyl, or 5- to 10-membered heterocyclyl;
each R^{a} and R^{b} is independently selected from H, halogen, OR′, OC(O)R′, NR′₂, C(O)R′, C(O)OR′, CN, NO₂, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl, wherein R′ is independently selected from H, C₁₋₆ alkyl, C₁₋₆ haloalkyl, C₆₋₁₂ aryl, (C₆₋₁₂)aryl(C₁₋₆)alkyl, and 5- to 10-membered heterocyclyl;
Y is selected from H, OR′, OC(O)R′, NR′₂, NR′₃, C(O)R′, C(O)OR′, OC(O)NR′₂, C(O)NR′₂, SR′, SOR′, SO₂R′, C₃₋₇ cycloalkyl, C₆₋₁₂ aryl, and 5- to 10-membered heterocyclyl, wherein each R′ is independently selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl;
p represents 0, 1, 2, 3, 4, 5, or 6; and
q represents 0, 1, 2, or 3;
   for use in the prevention or treatment of infections caused by RNA viruses.

Preferably, the compound of formula (I) is a compound of formula (I′)

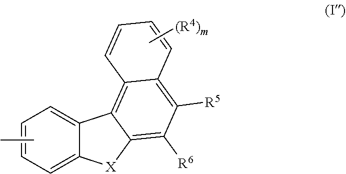

or a salt or solvate thereof, wherein n, m, R³, R⁴, R⁵, and R⁶ are as defined above, and
X is selected from O, S, NR⁷, and C(R⁸)(R⁹), wherein
   R⁷ is selected from H, C(O)R′, C(O)OR′, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl, optionally substituted, wherein R′ is independently selected from C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl;
   R⁸ and R⁹ are independently selected from H, halogen, OR′, OC(O)R′, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl, optionally substituted, wherein R′ is independently selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl; or R⁸ and R⁹ form, together with the carbon atom to which they are attached, a C═O group.

More preferably, the compound of formula (I) is a compound of formula (I″)

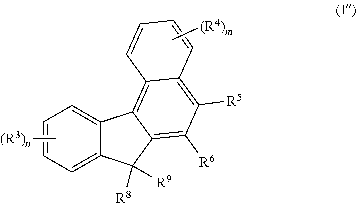

or a salt or solvate thereof, wherein n, m, R³, R⁴, R⁵, R⁶, R⁸ and R⁹ are as defined above.

The following particular and preferred embodiments apply to the compounds of formula (I), (I′), and (I″).

In a particular embodiment,
R¹ and R² are independently selected from H, halogen, OR′, OC(O)R′, C(O)R′, C(O)OR′, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl, optionally substituted, wherein R′ is independently selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl; or
R¹ and R² form, together with the carbon atom to which they are attached, a 5-membered carbocycle or heterocycle;
each R³ and R⁴ is independently selected from halogen, OR′, OC(O)R′, SR′, SOR′, SO₂R′, NR′₂, C(O)R′, C(O)OR′, CN, NO₂, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl, optionally substituted, wherein R′ is independently selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl; or two contiguous R³ or R⁴ groups form a benzene ring;
R⁵ is selected from H, halogen, OC(O)R′, SOR′, SO₂R′, C(O)R′, C(O)OR′, CN, NO₂, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl, optionally substituted, and a group of formula (A), wherein R′ is independently selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl;
R⁶ is selected from H, halogen, OR′, OC(O)R′, SR′, SOR′, SO₂R′, NR′₂, C(O)R′, C(O)OR′, CN, NO₂, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl, optionally substituted, wherein R′ is independently selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl;
n represents 0, 1, 2, 3, or 4; and
m represents 0, 1, 2, 3, or 4.

In a preferred embodiment, in the group of formula (A):
Z is selected from 5- to 10-membered heterocyclyl, O, S, and NR′, wherein R′ represents H, C₁₋₆ alkyl, C₁₋₆ haloalkyl, C₆₋₁₂ aryl, (C₆₋₁₂)aryl(C₁₋₆)alkyl, or 5- to 10-membered heterocyclyl;
each R^{a} and R^{b} is independently selected from H, halogen, OH, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl;
Y is selected from NR′₂, ⁺NR′₃, OR′, OC(O)NR′₂, C(O)NR′₂, and 5- to 10-membered heterocyclyl, wherein each R′ is independently selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl;
p represents 0, 1, 2, 3, 4, 5, or 6; and
q represents 0, 1, 2, or 3.

In a preferred embodiment, in the group of formula (A) q is 0. In another embodiment, each R^{a} and R^{b} is independently selected from H, OH, and C₁₋₆ alkyl; they are more preferably H. In a particular embodiment, q is 0 and R^{a} and R^{b} are independently selected from H, OH, and C₁₋₆ alkyl. Preferably, q is 0 and R^{a} and R^{b} are H.

In a preferred embodiment, in the group of formula (A):
Z is selected from 5- or 6-membered heterocyclyl, O, and NR′, wherein R′ represents H, C₁₋₆ alkyl, C₁₋₆ haloalkyl, C₆₋₁₂ aryl, (C₆₋₁₂)aryl(C₁₋₆)alkyl, or 5- to 10-membered heterocyclyl;
each R^{a} and R^{b} is independently selected from H, OH, and C₁₋₆ alkyl;
Y is selected from NR′₂ and 5- to 10-membered heterocyclyl, wherein each R′ is independently selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl;
p represents 0, 1, 2, 3, 4, 5, or 6; and
q represents O.

Preferably, in the group of formula (A) p is selected from 1, 2, 3, 4, 5, or 6, more preferably from 1, 2, 3, or 4.

Preferably, the C₁₋₆ alkyl, C₃₋₇ cycloalkyl, C₆₋₁₂ aryl, (C₆₋₁₂)aryl(C₁₋₆)alkyl, and 5- to 10-membered heterocyclyl groups, optionally substituted, can be substituted by halogen, OR″, OC(O)R″, SR″, SOR″, SO₂R″, NR″₂, C(O)R″, C(O)OR″, CN, NO₂, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl.

In one embodiment of the invention, R⁵ is selected from a group of formula (A), C₁₋₆ alkyl, C₃₋₇ cycloalkyl, C₁₋₆ haloalkyl, C₆₋₁₂ aryl, (C₆₋₁₂)aryl(C₁₋₆)alkyl, 5- to 10-membered heterocyclyl, optionally substituted, OC(O)R′, SOR′, SO₂R′, C(O)R′, C(O)OR′, wherein R′ is independently selected from C₁₋₆ alkyl, C₁₋₆ haloalkyl, C₆₋₁₂ aryl, (C₆₋₁₂)aryl(C₁₋₆)alkyl, and 5- to 10-membered heterocyclyl.

Preferably, the C₁₋₆ alkyl, C₃₋₇ cycloalkyl, C₆₋₁₂ aryl, (C₆₋₁₂)aryl(C₁₋₆)alkyl, and 5- to 10-membered heterocyclyl groups in R⁵ can be optionally substituted by halogen, OR″, OC(O)R″, SR″, SOR″, SO₂R″, NR″₂, C(O)R″, C(O)OR″, CN, NO₂, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl.

Preferably, R⁵ is a group of formula (A) as defined above.

In one embodiment, R⁶ is selected from H, halogen, OH, OR′, NR′₂, C₁₋₆ alkyl, and haloalkyl, wherein each R′ is independently selected from C₁₋₆ alkyl and C₁₋₆ haloalkyl. Preferably, R⁶ is H.

In one embodiment, each R³ and R⁴ is independently selected from halogen, OH, OR′, NR′₂, C₁₋₆ alkyl, and haloalkyl, wherein each R′ is independently selected from C₁₋₆ alkyl and C₁₋₆ haloalkyl; or two contiguous R³ or R⁴ groups form a benzene ring. Preferably, each R³ and R⁴ is independently selected from halogen, OH, OR′, C₁₋₆ alkyl, and C₁₋₆ haloalkyl, wherein R′ is selected from C₁₋₆ alkyl and C₁₋₆ haloalkyl.

In a particular embodiment, n and m are independently selected from 0, 1, and 2, preferably from 0 and 1. In one embodiment of the invention, n and m are 0.

In one embodiment, R⁶ is H and n and m are 0.

In another embodiment, R⁵ is a group of formula (A) as defined above and R⁶ is selected from H, halogen, OH, OR′, NR′₂, C₁₋₆ alkyl, and haloalkyl, wherein each R′ is independently selected from C₁₋₆ alkyl and C₁₋₆ haloalkyl; it is preferably H.

In a preferred embodiment, R⁵ is a group of formula (A) and R⁶ is H.

In another preferred embodiment, R⁵ is a group of formula (A), R⁶ is H, and n and m are 0.

In a particular embodiment, R⁸ and R⁹ are independently selected from H, C₁₋₆ alkyl, C₃₋₇ cycloalkyl, C₁₋₆ haloalkyl, and (C₆₋₁₂)aryl(C₁₋₆)alkyl, or R¹ and R² form, together with the carbon atom to which they are attached, a C═O group. Preferably, R¹ and R² form, together with the carbon atom to which they are attached, a C═O group.

In one embodiment, the compound of formula (I) is compound 1a

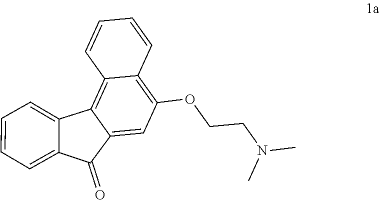 [SMILES: CN(C)CCOc1cc2c(c3ccccc13)-c1ccccc1C2=O]

or a salt or a solvate thereof.

In one embodiment of the invention, the RNA virus is selected from HIV-1 virus, HIV-2 virus, severe acute respiratory syndrome virus, hepatitis C virus, hepatitis A virus, hepatitis E virus, yellow fever virus, dengue virus, West Nile virus, poliovirus, influenza virus, Ebola virus, parainfluenza virus, rotavirus, chikungunya virus, rubella virus, and measles virus, among others.

Preferably, the RNA virus is HIV-1.

### Compounds of Formula (II)

In one aspect, the invention relates to a compound of formula (II)

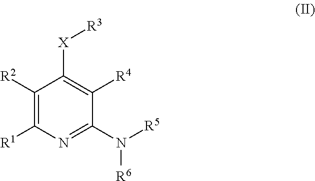

or a salt or a solvate thereof, wherein
R¹, R², and R⁴ are independently selected from H, halogen, OR′, OC(O)R′, SR′, SOR′, SO₂R′, NR′₂, C(O)R′, C(O)OR′, CN, NO₂, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl, optionally substituted, wherein R′ is independently selected from H, C₁₋₆ alkyl, C₁₋₆ haloalkyl, C₆₋₁₂ aryl, (C₆₋₁₂)aryl(C₁₋₆)alkyl, and 5- to 10-membered heterocyclyl;
X is selected from O, S, SO, and SO₂;
R³ is selected from C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl, optionally substituted; and
R⁵ and R⁶ are independently selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, 5- to 10-membered heterocyclyl, or R⁵ and R⁶ form, together with the nitrogen atom to which they are attached, an optionally substituted 5- to 10-membered heterocyclyl;
   for use in the prevention or treatment of infections caused by RNA viruses.

In a particular embodiment, R¹, R², and R⁴ are independently selected from H, halogen, OH, C₁₋₆ alkyl, and C₁₋₆ haloalkyl. Preferably, they are independently selected from H and halogen; more preferably halogen.

In one embodiment of the invention, X is selected from S, SO, and SO₂, preferably S.

In one embodiment, R³ is selected from C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl, optionally substituted with halogen, OR′, OC(O)R′, NR′₂, C(O)R′, C(O)OR′, CN, NO₂, SR′, SOR′, SO₂R′, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl, wherein R′ is independently selected from H, C₁₋₆ alkyl, C₁₋₆ haloalkyl, C₆₋₁₂ aryl, (C₆₋₁₂)aryl(C₁₋₆)alkyl, and 5- to 10-membered heterocyclyl. Preferably, R³ is C₆₋₁₂ aryl optionally substituted with halogen, OR′, OC(O)R′, NR′₂, C(O)R′, C(O)OR′, CN, NO₂, SR′, SOR′, SO₂R′, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl, wherein R′ is independently selected from H, C₁₋₆ alkyl, C₁₋₆ haloalkyl, C₆₋₁₂ aryl, (C₆₋₁₂)aryl(C₁₋₆)alkyl, and 5- to 10-membered heterocyclyl.

In a particular embodiment, R⁵ and R⁶ form, together with the nitrogen atom to which they are attached, a 5- to 10-membered heterocyclyl optionally substituted with halogen, OR′, OC(O)R′, NR′₂, C(O)R′, C(O)OR′, CN, NO₂, SR′, SOR′, SO₂R′, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl, wherein R′ is independently selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl.

In a preferred embodiment, R⁵ and R⁶ form, together with the nitrogen atom to which they are attached, a 5- to 7-membered heterocyclyl, preferably a 5- or 6-membered heterocycle, more preferably a heterocyclyl selected from pyrrolidine, piperidine, piperazine, morpholine, pyridine, pyrimidine, and pyrazine; preferably piperazine. More preferably, R⁵ and R⁶ form, together with the nitrogen atom to which they are attached, a heterocyclyl selected from pyrrolidine, piperidine, piperazine, morpholine, pyridine, pyrimidine, and pyrazine, even more preferably piperazine optionally substituted with halogen, OR′, OC(O)R′, NR′₂, C(O)R′, C(O)OR′, CN, NO₂, SR′, SOR′, SO₂R′, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl, wherein R′ is independently selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl.

More preferably:
R³ is selected from C₃₋₇ cycloalkyl, C₆₋₁₂ aryl, and 5- to 10-membered heterocyclyl, optionally substituted; and
R⁵ and R⁶ form, together with the nitrogen atom to which they are attached, an optionally substituted 5- to 10-membered heterocyclyl.

In one embodiment, the compound of formula (II) is compound 2a

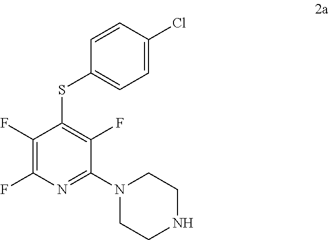 [SMILES: Fc1nc(N2CCNCC2)c(F)c(Sc2ccc(Cl)cc2)c1F]

or a salt or a solvate thereof.

In one embodiment of the invention, the RNA virus is selected from HIV-1 virus, HIV-2 virus, severe acute respiratory syndrome virus, hepatitis C virus, hepatitis A virus, hepatitis E virus, yellow fever virus, dengue virus, West Nile virus, poliovirus, influenza virus, Ebola virus, parainfluenza virus, rotavirus, chikungunya virus, rubella virus, and measles virus, among others.

Preferably, the RNA virus is HIV-1.

### Compounds of Formula (III)

In one aspect, the invention relates to a compound of formula (III)

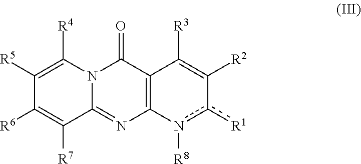

or a salt or a solvate thereof, wherein
R², R³, R⁴, R⁵, R⁶, and R⁷ are independently selected from H, halogen, OR′, OC(O)R′, C₁₋₆ alkyl, C₃₋₇ cycloalkyl, C₁₋₆ haloalkyl, C₆₋₁₂ aryl, (C₆₋₁₂)aryl(C₁₋₆)alkyl, 5- to 7-membered heterocyclyl, optionally substituted, SR′, SOR′, SO₂R′, NR′₂, C(O)R′, C(O)OR′, CN, and NO₂, wherein R′ is independently selected from H, C₁₋₆ alkyl, C₁₋₆ haloalkyl, C₆₋₁₂ aryl, (C₆₋₁₂)aryl(C₁₋₆)alkyl, and 5- to 7-membered heterocyclyl, optionally substituted;
R¹ is selected from NR′, O, S, H, halogen, OR′, OC(O)R′, C₁₋₆ alkyl, C₃₋₇ cycloalkyl, C₁₋₆ haloalkyl, C₆₋₁₂ aryl, (C₆₋₁₂)aryl(C₁₋₆)alkyl, 5- to 7-membered heterocyclyl, optionally substituted, SR′, SOR′, SO₂R′, NR′₂, C(O)R′, C(O)OR′, CN, and NO₂, wherein R′ is independently selected from H, C₁₋₆ alkyl, C₁₋₆ haloalkyl, C₆₋₁₂ aryl, (C₆₋₁₂)aryl(C₁₋₆)alkyl, and 5- to 7-membered heterocyclyl, optionally substituted;
R⁸ can be absent or selected from C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 7-membered heterocyclyl, optionally substituted; and
[symbol] represents a single bond or a double bond, such that one of the two bonds indicated as (N [symbol] C or C [symbol] R¹) represents a single bond and the other a double bond;
   for use in the prevention or treatment of infections caused by RNA viruses.

When the bond in position C [symbol] R¹ is a double bond, then R¹ is selected from NR′, O, and S, wherein R′ is independently selected from H, C₁₋₆ alkyl, C₁₋₆ haloalkyl, C₆₋₁₂ aryl, (C₆₋₁₂)aryl(C₁₋₆)alkyl, and 5- to 7-membered heterocyclyl, optionally substituted. When the bond in position C [symbol] R¹ is a single bond, then R¹ is selected from H, halogen, OR′, OC(O)R′, C₁₋₆ alkyl, C₃₋₇ cycloalkyl, C₁₋₆ haloalkyl, C₆₋₁₂ aryl, (C₆₋₁₂)aryl(C₁₋₆)alkyl, 5- to 7-membered heterocyclyl, optionally substituted, SR′, SOR′, SO₂R′, NR′₂, C(O)R′, C(O)OR′, CN, and NO₂, wherein R′ is independently selected from H, C₁₋₆ alkyl, C₁₋₆ haloalkyl, C₆₋₁₂ aryl, (C₆₋₁₂)aryl(C₁₋₆)alkyl, and 5- to 7-membered heterocyclyl, optionally substituted.

In a particular embodiment, R¹ is selected from NR′, O, S, OR′, SR′, SOR′, SO₂R′, NR′₂, and 5- to 7-membered heterocyclyl, wherein each R′ is independently selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 7-membered heterocyclyl, or two R′ groups form, together with the atom to which they are attached, a 5- to 7-membered heterocyclyl.

In one embodiment of the invention, R¹ is selected from NR⁹ and NR⁹R¹⁰, wherein R⁹ and R¹⁰ are independently selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, 5- to 7-membered heterocyclyl, or R⁹ and R¹⁰ form, together with the nitrogen atom to which they are attached, a 5- to 7-membered heterocyclyl.

In a preferred embodiment, the bond in position C [symbol] R¹ is a double bond, and R¹ represents NR⁹, wherein R⁹ is selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 7-membered heterocyclyl. More preferably, R¹ represents NH.

In another preferred embodiment, R¹ represents NR⁹R¹⁰, wherein R⁹ and R¹⁰ are independently selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 7-membered heterocyclyl. More preferably, R⁹ and R¹⁰ are independently selected from H and C₁₋₆ alkyl. More preferably, R⁹ and R¹⁰ are H.

In one embodiment, R² is selected from OR′, SR′, SOR′, and SO₂R′, wherein R′ is independently selected from C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 7-membered heterocyclyl, optionally substituted

In a preferred embodiment, R² is selected from OR′, SR′, SOR′, and SO₂R′, wherein R′ is independently selected from C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 7-membered heterocyclyl, optionally substituted with halogen, OR″, OC(O)R″, NR″₂, C(O)R″, C(O)OR″, CN, NO₂, SR″, SOR″, SO₂R″, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 7-membered heterocyclyl, wherein R″ is independently selected from H, C₁₋₆ alkyl, C₁₋₆ haloalkyl, C₆₋₁₂ aryl, (C₆₋₁₂)aryl(C₁₋₆)alkyl, and 5- to 7-membered heterocyclyl.

More preferably, R² is SO₂R′, wherein R′ is C₆₋₁₂ aryl optionally substituted with halogen, OR″, OC(O)R″, NR″₂, C(O)R″, C(O)OR″, CN, NO₂, SR″, SOR″, SO₂R″, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 7-membered heterocyclyl, wherein R″ is independently selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 7-membered heterocyclyl.

In one embodiment, R³, R⁴, R⁵, R⁶, and R⁷ are independently selected from H, halogen, OR′, C₁₋₆ alkyl, and C₁₋₆ haloalkyl, wherein R′ is independently selected from H, C₁₋₆ alkyl, and C₁₋₆ haloalkyl. In a particular embodiment, R³, R⁴, R⁵, R⁶, and R⁷ are H.

In one embodiment, R⁸ is absent.

In another embodiment, R⁸ is selected from C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 7-membered heterocyclyl. Preferably, R⁸ is selected from C₁₋₆ alkyl and C₃₋₇ cycloalkyl; preferably C₃₋₇ cycloalkyl.

According to one embodiment, when the bond in position N [symbol] C is a single bond, then R⁸ is selected from C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 7-membered heterocyclyl, more preferably it is selected from C₁₋₆ alkyl and C₃₋₇ cycloalkyl; even more preferably C₃₋₇ cycloalkyl. According to one embodiment of the invention, when the bond in position N [symbol] C is a double bond, then R⁸ is absent. According to another embodiment, when the bond in position N [symbol] C is a double bond, then R⁸ is selected from C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 7-membered heterocyclyl, more preferably it is selected from C₁₋₆ alkyl and C₃₋₇ cycloalkyl; even more preferably C₃₋₇ cycloalkyl, and in this case (when R⁸ is not absent and N [symbol] C is a double bond), the nitrogen atom substituted with one of these groups is positively charged.

In one embodiment:
R¹ is selected from NR′, O, S, OR′, SR′, SOR′, SO₂R′, NR′₂, and 5- to 7-membered heterocyclyl, wherein each R′ is independently selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 7-membered heterocyclyl, or two R′ groups form, together with the atom to which they are attached, a 5- to 7-membered heterocyclyl;
R² is selected from OR′, SR′, SOR′, and SO₂R′, wherein R′ is independently selected from C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 7-membered heterocyclyl, optionally substituted with halogen, OR″, OC(O)R″, NR″₂, C(O)R″, C(O)OR″, CN, NO₂, SR″, SOR″, SO₂R″, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 7-membered heterocyclyl, wherein R″ is independently selected from H, C₁₋₆ alkyl, C₁₋₆ haloalkyl, C₆₋₁₂ aryl, (C₆₋₁₂)aryl(C₁₋₆)alkyl, and 5- to 7-membered heterocyclyl.

In a particular embodiment:
R¹ represents NR⁹ or NR⁹R¹⁰, wherein R⁹ and R¹⁰ are independently selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, 5- to 7-membered heterocyclyl, or R⁹ and R¹⁰ form, together with the nitrogen atom to which they are attached, a 5- to 7-membered heterocyclyl;
R² is SO₂R′, wherein R′ is C₆₋₁₂ aryl optionally substituted with halogen, OR″, OC(O)R″, NR″₂, C(O)R″, C(O)OR″, CN, NO₂, SR″, SOR″, SO₂R″, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 7-membered heterocyclyl, wherein R″ is independently selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 7-membered heterocyclyl.

In one embodiment, the compound of formula (III) is compound 2b

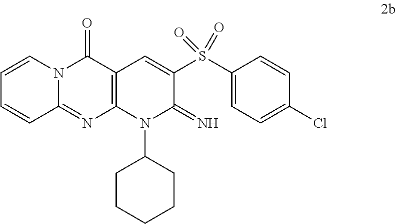 [SMILES: N=c1c(S(=O)(=O)c2ccc(Cl)cc2)cc2c(=O)n3ccccc3nc2n1C1CCCCC1]

or a salt or a solvate thereof.

In one embodiment of the invention, the RNA virus is selected from HIV-1 virus, HIV-2 virus, severe acute respiratory syndrome virus, hepatitis C virus, hepatitis A virus, hepatitis E virus, yellow fever virus, dengue virus, West Nile virus, poliovirus, influenza virus, Ebola virus, parainfluenza virus, rotavirus, chikungunya virus, rubella virus, and measles virus, among others.

Preferably, the RNA virus is HIV-1.

The following non-limiting examples seek to illustrate the present invention and must not be interpreted as limiting the scope thereof.

### EXAMPLES

Materials and Methods

Compounds.

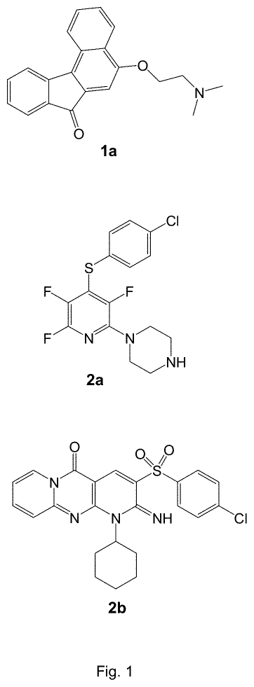

Compound 1a was obtained through Sigma-Aldrich, St. Louis, USA (MyriaScreen compound library). Compound 2a was obtained through Sigma-Aldrich (St. Louis, USA). Compound 2b was obtained through AKosGmbH (Steinen, Germany). Neomycin B used as control in FA experiments was acquired through Sigma-Aldrich (St. Louis, USA) (FIG. 1).

RNA, DNA, Peptide, and Protein Samples.

The composition and preparation of the following species have been previously described in detail in the state of the art (S. Prado et al., Biochem Pharmacol 107 (2016) 14-28; Gonzalez-Bulnes et al., Angewandte Chemie-International Edition 52(50) (2013) 13405-13409): 234-nucleotide RNA sequence RRE, RNA oligonucleotides corresponding to subdomain IIB IIBh, IIBh-19ap, and IIBh-23fl, Escherichia coli RNAtCys, 26-nucleotide self-complementary DNA oligonucleotide LTRd, complete Rev protein, and FITCfrevp-labeled Rev34-50 peptide. RRE and Rev were used in EMSA assays, and non-labeled IIBh was used in NMR and FA spectroscopy experiments. In FA assays, frevp was used. IIBh-19ap and IIBh-23fl contained 2-aminopurine instead of A in the unpaired A19 nucleotide of the loop and FITC covalently attached to the extra-helical U23 nucleotide of the loop, respectively, and were used in fluorescence intensity assays. RNAtCys and LTRd were used as RNA and DNA specificity controls in these fluorescence intensity experiments.

Fluorescence Anisotropy (FA).

These experiments were performed and analyzed as described in the state of the art (Prado et al., Biochem Pharmacol 107 (2016) 14-28; Gonzalez-Bulnes et al., Angewandte Chemie-International Edition 52(50) (2013) 13405-13409; Luedtke et al., Biopolymers 70(1) (2003) 103-19), using 96-well plates and Victor X3 or Victor X5 plate readers (Perkin Elmer, Waltham USA). The assays used 10 nM of frevp and 60 nM of IIBh, and included one positive control (a mixture of IIBh and frevp) and two negative controls (frevp and a mixture of IIBh, frevp, and neomycin B). IIBh-frevp inhibition was only considered to exist when anisotropy reached the expected minimum value at the highest inhibitor concentrations. To ensure that there would not be spectral superposition with FITC, anisotropy data was also collected for each of the compounds. None of them emitted fluorescence under the assay conditions. These experiments were repeated three times for each compound.

Electrophoretic Mobility Shift Assays (EMSA).

These experiments used 78 nM of complete RRE, 1.32 μM of complete Rev, and increasing concentrations of each compound, and were carried out and analyzed as described previously in the state of the art (Prado et al., Biochem Pharmacol 107 (2016) 14-28; Fang et al., Cell 155(3) (2013) 594-605). The IC₅₀ values were determined by measuring the area and the intensity of the band corresponding to free RRE. These experiments were performed at least three times for each compound.

Fluorescence Intensity.

Depending on the absorption spectra of the compounds, these experiments measured association with IIBh-19ap or IIBh-23fl RNA sequences labeled with 2-aminopurine and fluorescein in residues A19 and U23 of the IIB loop, respectively (Prado et al., Biochem Pharmacol 107 (2016) 14-28), and were carried out in a SPECTRA GEMINI XPS plate reader (Molecular Devices, Sunnyvale, USA) or a Victor X5 plate reader (Perkin Elmer). The IIB RNA concentration in these assays was 100 nM, and the specificity of the interactions with respect to RNA and DNA was evaluated by duplicating the experiments in the presence of a 100-fold molar excess (10 μM) of RNAtCys or LTRd DNA duplex. All the fluorescence intensity experiments were performed at least three times for each compound.

NMR Spectroscopy.

The NMR spectra were acquired in a 500 MHz Bruker Avance spectrometer and analyzed with Topspin 3.5 (Bruker Biospin, Billerica, USA). The interaction between the tested compounds and IIBh RNA samples dissolved at a concentration of 40-60 μM were monitored as described previously in the state of the art (Prado et al., Biochem Pharmacol 107 (2016) 14-28; Gonzalez-Bulnes et al., Angewandte Chemie-International Edition 52(50) (2013) 13405-13409), using one-dimensional and two-dimensional experiments (TOCSY) at increasing ligand:RNA molar ratios.

Plasmids, Virus, and Cells for Ex Vivo Assays.

Vectors pNL4.3-Luc and pNL4.3-Ren were generated by cloning luciferase and Renilla genes, respectively, in the nef site of HIV-1 proviral plasmid pNL4.3 (Adachi et al., J Virol 59(2) (1986) 284-91), as described previously in the state of the art (Garcia-Perez et al., J Med Virol 79(2) (2007) 127-37). MT-2 cells (Harada et al., Science 229(4713) (1985) 563-6) and 293T cells (American Type Culture Collection, Rockville, USA) were cultured as described previously (Prado et al., Biochem Pharmacol 107 (2016) 14-28).

Anti-HIV-1 Activity and Cell Toxicity.

The methodology used for performing and analyzing these experiments has been described previously (Prado et al., Biochem Pharmacol 107 (2016) 14-28; Gonzalez-Bulnes et al., Angewandte Chemie-International Edition 52(50) (2013) 13405-13409). Briefly, infectious supernatants were obtained from 293T cells transfected with the pNL4.3-Ren plasmid. MT-2 cells were infected with these supernatants in the presence of the compounds, and the anti-HIV activity was quantified 48 h after infection by determining luciferase activity in the cell lysates. Cell viability was evaluated in cells treated with the same compound concentrations using the CellTiter-Glo assay (Promega). The antiviral activity and cell toxicity results represent the average of at least three independent experiments.

Cell Transfection Assays.

MT-2 cells were transfected with plasmids containing a luciferase reporter gene the expression of which was under the control of the complete HIV-1 (NL4.3-luc) or HIV-1 LTR promoter (LTR-Luc). After transfection, the cells were treated with different compound concentrations, and the luciferase activity in the cell lysates was quantified 48 h later (Prado et al., Biochem Pharmacol 107 (2016) 14-28; Gonzalez-Bulnes et al., Angewandte Chemie-International Edition 52(50) (2013) 13405-13409).

HIV-1 RNA Processing Analysis.

MT-2 cells, previously treated for 72 or 96 hours with two different concentrations (5 and 10 μM) of compound 1a or 2a, were transfected with a pNL4.3 plasmid. Total cellular RNA was then isolated, treated with DNase I, and reverse transcribed as described in the state of the art (Prado et al., Biochem Pharmacol 107 (2016) 14-28). The unprocessed, semi-processed, and completely processed HIV-1 RNA transcripts were quantified by means of qPCR in relation to an untreated control obtained, using primers described by Mohammadi et al. (PLoS Pathog 9(1) (2013) e1003161), and GAPDH as endogenous control.

Results

Inhibition of the Interaction Between Subdomain IIB of RRE and Rev₃₄₋₅₀, and Inhibition of the Complete RRE-Rev Complex

The capacity of the compounds to inhibit IIB-Rev₃₄₋₅₀ interaction was evaluated by means of an assay based on the detection, by means of fluorescence anisotropy (FA), of the shift of an FITC-labeled Rev₃₄₋₅₀ peptide from its binding site in subdomain IIB of RRE. The capacity of the compounds to inhibit the formation of the RRE-Rev complex was evaluated by means of an electrophoretic mobility shift assay (EMSA).

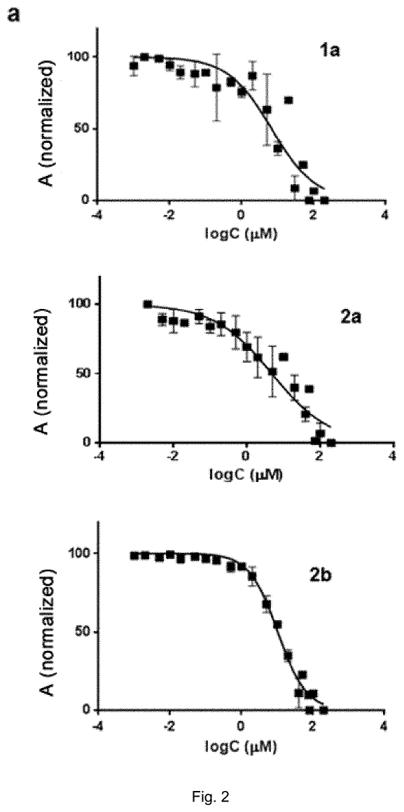

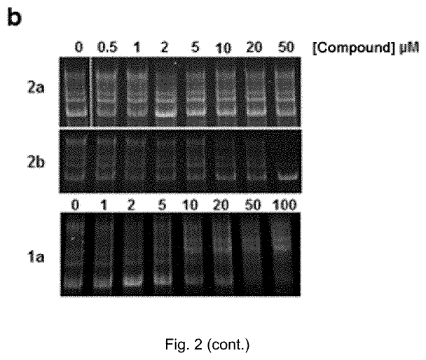

It was found that compounds 1a, 2a, and 2b interfere with both IIB-Rev₃₄₋₅₀ interaction and the formation of the complete RRE-Rev complex (Table 1, FIG. 2). The most potent compound was 2a (with an IC₅₀ between 4.7 and 4.8 μM in both FA and EMSA experiments), followed by 2b (10.3-13.7 μM). The increase in the concentrations of these two compounds increased the intensity of the band corresponding to free RRE in the EMSA assays. This indicates the inhibition of the full-length RRE-Rev complex which typically has several electrophoretic bands corresponding to complexes with different number of associated Rev monomers (FIG. 2b). Compound 1a blocked the interaction between subdomain IIB and Rev34-50 with an IC₅₀ of 6.7 μM), and promoted the formation of RRE-Rev complexes with an unusual molecular weight at concentrations greater than 10 μM (FIG. 2b).

TABLE 1

| | IC₅₀ | IC₅₀ |
|---|---|---|
| Compound^{a} | IIB-Rev₃₄₋₅₀ (M · 10⁶) | RRE-Rev (M · 10⁶) |
| 1a | 6.7 | ^{b} |
| | (4.2-10.5; 0.8398) | |
| 2a | 4.8 | 4.7 |
| | (3.2-7.1; 0.8910) | (2.2-7.3; 0.9156) |
| 2b | 10.3 | 13.7 |
| | (9.2-11.5; 0.9843) | (5.6-21.8; 0.9270) |

^{a}The IC₅₀ values of IIBₕ-Rev₃₄₋₅₀ were obtained by means of FA experiments using 60 nM IIBₕ and 10 nM frevp, and the IC₅₀ values of RRE-Rev were measured by means of EMSA with 78 nM RRE and 1.32 μM Rev. The table shows the IC₅₀ values obtained by means of two (EMSA) or three (FA) independent experiments; the 95% confidence intervals and R² coefficients are shown in parentheses.
^{b} At concentrations 10 μM, 1a induced an unusual accumulation of RRE-Rev complexes of intermediate molecular weight instead of free RRE. This prevented the determination of the IC₅₀ value of RRE-Rev.

RNA-Binding Properties

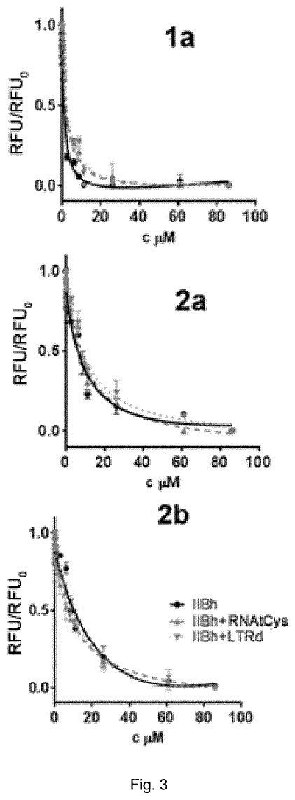

To verify if the compounds inhibit the interaction between RRE and Rev by binding to RRE RNA, association with subdomain IIB of the RRE was measured with fluorescence intensity experiments, using the IIBh-19ap or IIBh-23fl RNA sequences containing 2-aminopurine and fluorescein probes in unpaired A19 and U23 residues of loop IIB, respectively (Prado et al., Biochem Pharmacol 107 (2016) 14-28) (FIG. 3). The specificity of the interaction was evaluated by duplicating the experiments in the presence of a 100-fold molar excess of RNAtCys. Likewise, specificity with respect to a double-stranded DNA was evaluated by further performing the experiments with a 100-fold molar excess of a DNA duplex with 26 base pairs (identified as LTRd) containing NF-κB and Sp-1 transcription factor-binding sites (Prado et al., Biochem Pharmacol 107 (2016) 14-28). These two factors bind to the LTR promoter of HIV-1 DNA and are essential for virus replication (Jones et al., AnnuRevBiochem 63 (1994) 717-43).

Compounds 1a, 2a, and 2b associated with subdomain IIB of the RRE of the RNA. The compound binding the strongest was 1a with a K_{d} of 1.4 μM, followed by 2a and 2b (10.8-12.1 μM) (Table 2, FIG. 3). The interaction was specific for 2a and 2b, as indicated in the binding curves obtained in the presence of a 100-fold molar excess of RNAtCys or LTRd. Compound 1a exhibited moderate specificity.

TABLE 2

| Comp.^{a} | K_{d}(IIBₕ) (M · 10⁶) | K_{d}(IIBₕ + ₜRNA) (M · 10⁶) | specificity IIB/RNA $\frac{{K}_{d} ( I I {B}_{h} )}{{K}_{d} ( I I {B}_{h} + {}_{t} RNA )}$ | K_{d}(IIBₕ + LTR_{d}) (M · 10⁶) | specificity IIB/DNA $\frac{{K}_{d} ( I I {B}_{h} )}{{K}_{d} ( I I {B}_{h} + L T {R}_{d} )}$ |
|---|---|---|---|---|---|
| 1a | 1.4 | 2.4 | 0.58 | 2.8 | 0.50 |
| | (1.0-1.8; 0.9849) | (1.5-3.2; 0.9830) | | (1.7-3.9; 0.9812) | |
| 2a | 10.8 | 9.0 | 1.20 | 9.2 | 1.17 |
| | (1.6-20.0; 0.9529) | (5.2-12.9; 0.9055) | | (5.9-12.5; 0.9397) | |
| 2b | 12.1 | 11.4 | 1.06 | 10.2 | 1.19 |
| | (6.0-18.1; 0.8841) | (0.8-21.9; 0.9547) | | (7.2-13.2; 0.9598) | |

^{a}For each compound, the table shows disassociation equilibrium constants of IIBh (Kd) in the absence (IIBh) and presence of RNAtCys (IIBh + ₜRNA) or DNA duplex LTRd (IIBh + LTR_{d}).
The specificity of the interaction was quantified by calculating the K_{d}(IIBₕ)/K_{d}(IIBₕ + ₜRNA) and K_{d}(IIBₕ)/K_{d}(IIBₕ + LTR_{d}) quotients.
Interactions with specificity ratios close to 1 or > 1 are specific, whereas those with ratios <<1 are non-specific.
All the compounds were analyzed using IIBₕ-19ap.
The table shows the K_{d} values obtained with two independent experiments; the 95% confidence intervals and R² coefficients are indicated in parentheses.

NMR spectroscopy is then used for identifying the binding sites in the IIBₕ fork of the compounds. The NMR results were in line with the fluorescence observations: 1a, which showed higher affinity for IIBₕ in the fluorescence assay, widened or altered the chemical shift of IIBh resonances at low drug:RNA molar ratios. This compound altered the signals of residues C₉, A₁₉, C₂₀, and U₂₃ comprising the Rev binding site, but also disrupted the signals of the double-helix stems flanking the loop. 2a and 2b required a higher drug:RNA molar ratio to induce changes in the signals of IIBh RNA. The binding of 2a affected the signals of residues located in the stems flanking the internal loop, whereas 2b, the compound showing a higher K_{d} in the fluorescence assay, only induced the widening of signals of residue U₂₃ of the loop at a high drug:RNA molar ratio.

Antiretroviral Activity and Cell Toxicity

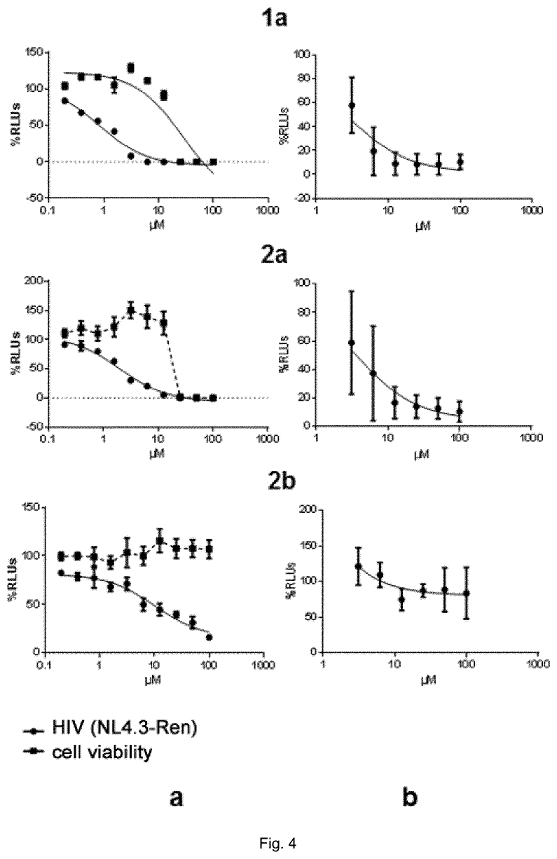

Compounds 1a, 2a, and 2b exhibited antiretroviral activity in a cell infection assay. The most potent inhibitor was 1a, with a submicromolar EC₅₀, value (830 nM), followed by 2a (EC₅₀=2.0 μM), and 2b (EC₅₀=10.2 μM) (Table 3 and FIG. 4a).

The cytotoxic concentrations of the compounds were also measured and compared with the antiviral concentrations obtained in the infection experiments. The CC₅₀ values were 34-, 22-, and >10-fold greater than the antiviral concentrations for 1a, 2a, and 2b (Table 3 and FIG. 4a).

TABLE 3

| | EC₅₀ of | EC₅₀ of | EC₅₀ of | |
|---|---|---|---|---|
| | infection, | transfection, | transfection, | |
| | HIV | HIV | LTR | CC₅₀ |
| Comp.^{a} | (M · 10⁶) | (M · 10⁶) | (M · 10⁶) | (M · 10⁶) |
| 1a | 0.83 | 2.5 | >12.5 < 25 | 28.1 |
| | (0.58-1.2; | (1.2-4.3; | | (12.0-65.7; |
| | 0.9707) | 0.5344) | | 0.9124) |
| 2a | 2.0 | 3.3 | 4.4 | 43.9 |
| | (1.5-2.6; | (1.3-6.7; | (3.4-5.8; | (11.5-168; |
| | 0.9809) | 0.4484) | 0.9323) | 0.7333) |
| 2b | 10.2 | >100 | n/d | >100 |
| | (5.9-17.6; | | | |
| | 0.9193) | | | |

^{a}The confidence intervals and R² coefficients are indicated in parentheses;
n/d: not determined

Location of the Antiviral Targets

In order to identify the target or the set of targets of the tested compounds, an assay based on the transfection of a full-length HIV-1 vector is performed. In this experiment, the entry, reverse transcription, and DNA integration phases of the viral cycle are sidestepped, such that only transcriptional or post-transcriptional events which can be blocked by the inhibitor under study are produced. When the compounds were evaluated with this transfection assay, compounds 1a and 2a showed low EC₅₀ values ranging between 2.5 and 3.3 μM (Table 3 and FIG. 4b). This result indicated that these compounds act in transcriptional or post-transcriptional processes of the viral cycle. In contrast, 2b was inactive in the transfection assay at concentrations lower than 100 μM. Given that this compound had an EC₅₀ value of 10.2 μM in the infection experiment, this result suggests that 2b acts mainly in the pre-transcriptional processes of the viral cycle in a cellular context.

Inhibition of HIV-1 Transcription and Rev Function

The RRE-Rev ribonucleoprotein allows unprocessed or partially processed viral transcripts to be transported into the cytoplasm, an essential post-transcriptional process required by the virus to complete its replication cycle. Compounds 1a and 2a altered IIB-Rev₃₄₋₅₀ and full-length RRE-Rev interactions at low concentrations (Table 1 and FIG. 2), recognized subdomain IIB of RRE with a high or moderate specificity in fluorescence experiments (Table 2 and FIG. 3), and exhibited lower EC₅₀ values in the HIV-1 transfection assay (Table 3 and FIG. 4b). In contrast, compound 2b inhibited IIB-Rev₃₄₋₅₀ and RRE-Rev complexes at higher concentrations, bound to subdomain IIB of the RRE with lower affinity (Tables 1 and 2 and FIG. 3), and exhibited less activity in the HIV-1 transfection assay (Table 3 and FIG. 4b).

The antiviral mechanism of compounds 1a and 2a was examined with additional cellular assays. First, whether or not they exhibited an effect on viral transcription was evaluated by means of determining their inhibitory activity in an experiment based on the transfection of a plasmid encoding a luciferase gene the expression of which depends on the LTR promoter of the virus (Hazen et al., Proc Natl Acad Sci USA 87(20) (1990) 7861-5). 1a exhibited a weaker inhibition of LTR-dependent expression in relation to the activity detected in the complete HIV-1 transfection assay, whereas 2a inhibited LTR-dependent expression with an EC₅₀ value similar to those measured in the HIV-1 infection and transfection experiments (Table 3). These results suggest that, although the antiviral activity of 1a is independent of LTR-dependent transcription, the effect of 2a may be due to LTR-mediated transcription inhibition.

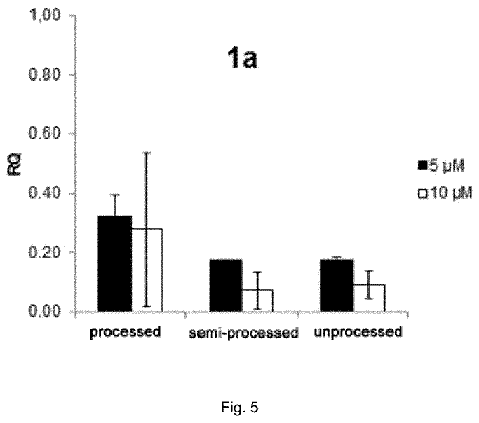

Whether or not compounds 1a and 2a acted in the RRE-Rev system in a cellular context was then verified. After transfecting the cells with a full-length proviral vector, the amounts of unprocessed, semi-processed, and completely processed HIV-1 RNA transcripts were quantified by means of RT-qPCR in the absence and presence of each compound (Prado et al., Biochem Pharmacol 107 (2016) 14-28). Given that Rev indirectly puts the splicing process at a disadvantage by transporting unprocessed or semi-processed viral transcripts from the nucleus to the cytoplasm, the blocking of the RRE-Rev system should increase the levels of completely processed species and reduce the amount of unprocessed or semi-processed HIV-1 transcripts. This effect was clearly observed when the cells were exposed to compound 1a at concentrations of 5 and 10 μM (FIG. 5). A significant reduction in the levels of viral transcripts in relation to untreated cells was also detected in the presence of this inhibitor. In contrast, the same concentration of 2a did not induce clear changes in the patterns of unprocessed viral transcripts with respect to processed viral transcripts, and reduced the amount of viral transcripts to a lesser extent with respect to 1a.

1. A method for the treatment of infections caused by RNA viruses, said method comprising administering to a patient in need of such treatment a therapeutically effective amount of a compound of formula (I): 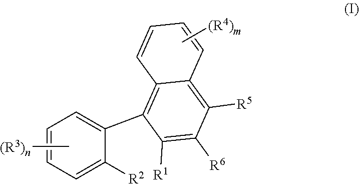
or a salt or solvate thereof, wherein
R¹ and R² are independently selected from H, halogen, OR′, OC(O)R′, C(O)R′, C(O)OR′, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl, optionally substituted, wherein R′ is independently selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl; or
R¹ and R² form, together with the carbon atom to which they are attached, a 5-membered carbocycle or heterocycle;
each R³ and R⁴ is independently selected from halogen, OC(O)R′, SOR′, SO₂R′, C(O)R′, C(O)OR′, CN, NO₂, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl, optionally substituted, and a group of formula (A), wherein R′ is independently selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl; or two contiguous R³ or R⁴ groups form a benzene ring;
R⁵ is selected from H, halogen, OC(O)R′, SOR′, SO₂R′, C(O)R′, C(O)OR′, CN, NO₂, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl, optionally substituted, and a group of formula (A), wherein R′ is independently selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl;
R⁶ is selected from H, halogen, OC(O)R′, SOR′, SO₂R′, C(O)R′, C(O)OR′, CN, NO₂, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl, optionally substituted, and a group of formula (A), wherein R′ is independently selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl;
n represents 0, 1, 2, 3, or 4; and
m represents 0, 1, 2, 3, or 4;
wherein at least one of R³, R⁴, R⁵, and R⁶ is a group of formula (A) 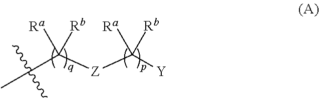
wherein
Z is selected from 5- to 10-membered heterocyclyl, 0, 5, and NR′, wherein R′ represents H, C₁₋₆ alkyl, C₃₋₇ cycloalkyl, C₁₋₆ haloalkyl, C₆₋₁₂ aryl, (C₆₋₁₂)aryl(C₁₋₆)alkyl, or 5- to 10-membered heterocyclyl;
each R^{a} and R^{b} is independently selected from H, halogen, OR′, OC(O)R′, NR′₂, C(O)R′, C(O)OR′, CN, NO₂, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl, wherein R′ is independently selected from H, C₁₋₆ alkyl, C₁₋₆ haloalkyl, C₆₋₁₂ aryl, (C₆₋₁₂)aryl(C₂₋₆)alkyl, and 5- to 10-membered heterocyclyl;
Y is selected from H, OR′, OC(O)R′, NR′₂, ⁺NR′₃, C(O)R′, C(O)OR′, OC(O)NR′₂, C(O)NR′₂, SR′, SOR′, SO₂R′, C₃₋₇ cycloalkyl, C₆₋₁₂ aryl, and 5- to 10-membered heterocyclyl, wherein each R′ is independently selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl;
p represents 0, 1, 2, 3, 4, 5, or 6; and
q represents 0, 1, 2, or 3.

2. (canceled)

3. The method according to claim 1, wherein the RNA virus is HIV-1.

4. The method according to claim 1, wherein the compound of formula (I) is a compound of formula (I′): 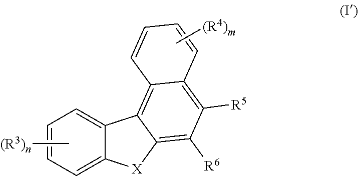
or a salt or solvate thereof, wherein
X is selected from O, S, NR⁷ and C(R⁸)(R⁹), wherein
R⁷ is selected from H, C(O)R′, C(O)OR′, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl, optionally substituted, wherein R′ is independently selected from C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl;
R⁸ and R⁹ are independently selected from H, halogen, OR′, OC(O)R′, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl, optionally substituted, wherein R′ is independently selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl; or
R⁸ and R⁹ form, together with the carbon atom to which they are attached, a C═O group;
R³, R⁴, R⁵, R⁶, n, and m are as defined in claim 1.

5. The method according to claim 1, wherein the compound of formula (I) is a compound of formula (I″): 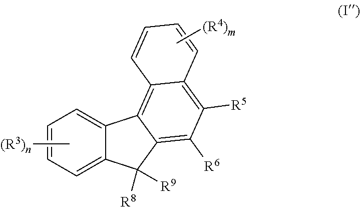
or a salt or solvate thereof, wherein
R⁸ and R⁹ are independently selected from H, halogen, OR′, OC(O)R′, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl, optionally substituted, wherein R′ is independently selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl; or
R⁸ and R⁹ form, together with the carbon atom to which they are attached, a C═O group;
R³, R⁴, R⁵, R⁶, n, and m are as defined in claim 1.

6. (canceled)

7. The method according to claim 1, wherein R⁵ is a group of formula (A): 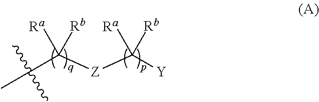
wherein
Z is selected from 5- to 10-membered heterocyclyl, O, S, and NR′, wherein R′ represents H, C₁₋₆ alkyl, C₃₋₇ cycloalkyl, C₁₋₆ haloalkyl, C₆₋₁₂ aryl, (C₆₋₁₂)aryl(C₁₋₆)alkyl, or 5- to 10-membered heterocyclyl;
each R^{a} and R^{b} is independently selected from H, halogen, OR′, OC(O)R′, NR′₂, C(O)R′, C(O)OR′, CN, NO₂, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl, wherein R′ is independently selected from H, C₁₋₆ alkyl, C₁₋₆ haloalkyl, C₆₋₁₂ aryl, (C₆₋₁₂)aryl(C₁₋₆)alkyl, and 5- to 10-membered heterocyclyl;
Y is selected from H, OR′, OC(O)R′, NR′₂, ⁺NR′₃, C(O)R′, C(O)OR′, OC(O)NR′₂, C(O)NR′₂, SR′, SOR′, SO₂R′, and 5- to 10-membered heterocyclyl, wherein each R′ is independently selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl;
p represents 0, 1, 2, 3, 4, 5, or 6; and
q represents 0, 1, 2, or 3.

8. The method according to claim 7, wherein
Z is selected from 5- to 10-membered heterocyclyl, O, S, and NR′, wherein R′ represents H, C₁₋₆ alkyl, C₁₋₆ haloalkyl, C₆₋₁₂ aryl, (C₆₋₁₂)aryl(C₁₋₆)alkyl, or 5- to 10-membered heterocyclyl;
each R^{a} and R^{b} is independently selected from H, halogen, OH, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl;
Y is selected from NR′₂, ⁺NR′₃, OR′, OC(O)NR′₂, C(O)NR′₂, and 5- to 10-membered heterocyclyl, wherein each R′ is independently selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl;
p represents 0, 1, 2, 3, 4, 5, or 6; and
q represents 0, 1, 2, or 3.

9. (canceled)

10. (canceled)

11. (canceled)

12. (canceled)

13. (canceled)

14. (canceled)

15. (canceled)

16. (canceled)

17. The method according to claim 1, wherein the compound of formula (I) is: 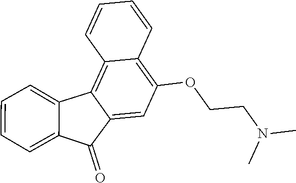 [SMILES: CN(C)CCOc1cc2c(c3ccccc13)-c1ccccc1C2=O]
or a salt or a solvate thereof.

18. A method for the treatment of infections caused by RNA viruses, said method comprising administering to a patient in need of such treatment a therapeutically effective amount of a compound of formula (II): 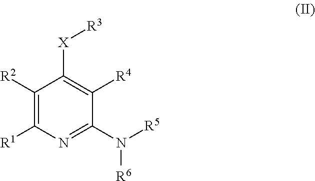
or a salt or a solvate thereof, wherein
R¹, R², and R⁴ are independently selected from H, halogen, OR′, OC(O)R′, SR′, SOR′, SO₂R′, NR′₂, C(O)R′, C(O)OR′, CN, NO₂, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl, optionally substituted, wherein R′ is independently selected from H, C₁₋₆ alkyl, C₁₋₆ haloalkyl, C₆₋₁₂ aryl, (C₆₋₁₂)aryl(C₁₋₆)alkyl, and 5- to 10-membered heterocyclyl;
X is selected from O, S, SO, and SO₂;
R³ is selected from C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl, optionally substituted; and
R⁵ and R⁶ are independently selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, 5- to 10-membered heterocyclyl, or R⁵ and R⁶ form, together with the nitrogen atom to which they are attached, an optionally substituted 5- to 10-membered heterocyclyl.

19. (canceled)

20. The method according to claim 18, wherein the RNA virus is HIV-1.

21. The method according to claim 18, wherein R¹, R², and R⁴ are independently selected from H, halogen, OH, C₁₋₆ alkyl, and C₁₋₆ haloalkyl.

22. The method according to claim 18, wherein X is selected from 5, SO, and SO₂, preferably S.

23. The method according to claim 18, wherein R³ is selected from C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl, optionally substituted with halogen, OR′, OC(O)R′, NR′₂, C(O)R′, C(O)OR′, CN, NO₂, SR′, SOR′, SO₂R′, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl, wherein R′ is independently selected from H, C₁₋₆ alkyl, C₁₋₆ haloalkyl, C₆₋₁₂ aryl, (C₆₋₁₂)aryl(C₁₋₆)alkyl, and 5- to 10-membered heterocyclyl.

24. (canceled)

25. The method according to claim 18, wherein R⁵ and R⁶ form, together with the nitrogen atom to which they are attached, a 5- to 10-membered heterocyclyl optionally substituted with halogen, OR′, OC(O)R′, NR′₂, C(O)R′, C(O)OR′, CN, NO₂, SR′, SOR′, SO₂R′, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl, wherein R′ is independently selected from H, C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl.

26. The method according to claim 18, wherein the compound of formula (II) is: 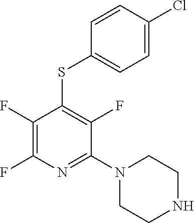 [SMILES: Fc1nc(N2CCNCC2)c(F)c(Sc2ccc(Cl)cc2)c1F]
or a salt or a solvate thereof.

27. A method for the treatment of infections caused by RNA viruses, said method comprising administering to a patient in need of such treatment a therapeutically effective amount of a compound of formula (III): 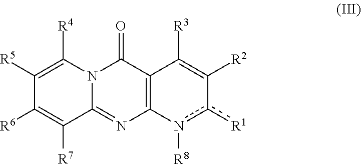
or a salt or a solvate thereof, wherein
R², R³, R⁴, R⁵, R⁶, and R⁷ are independently selected from H, halogen, OR′, OC(O)R′, C₁₋₆ alkyl, C₃₋₇ cycloalkyl, C₁₋₆ haloalkyl, C₆₋₁₂ aryl, (C₆₋₁₂)aryl(C₁₋₆)alkyl, 5- to 7-membered heterocyclyl, optionally substituted, SR′, SOR′, SO₂R′, NR′₂, C(O)R′, C(O)OR′, CN, and NO₂, wherein R′ is independently selected from H, C₁₋₆ alkyl, C₁₋₆ haloalkyl, C₆₋₁₂ aryl, (C₆₋₁₂)aryl(C₁₋₆)alkyl, and 5- to 7-membered heterocyclyl, optionally substituted;
R¹ is selected from NR′, O, S, H, halogen, OR′, OC(O)R′, C₁₋₆ alkyl, C₃₋₇ cycloalkyl, C₁₋₆ haloalkyl, C₆₋₁₂ aryl, (C₆₋₁₂)aryl(C₁₋₆)alkyl, 5- to 7-membered heterocyclyl, optionally substituted, SR′, SOR′, SO₂R′, NR′₂, C(O)R′, C(O)OR′, CN, and NO₂, wherein R′ is independently selected from H, C₁₋₆ alkyl, C₁₋₆ haloalkyl, C₆₋₁₂ aryl, (C₆₋₁₂)aryl(C₁₋₆)alkyl, and 5- to 7-membered heterocyclyl, optionally substituted;
R⁸ can be absent or selected from C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 7-membered heterocyclyl, optionally substituted; and [symbol]
represents a single bond or a double bond, such that one of the two bonds indicated as (N [symbol] C or C [symbol] R¹) represents a single bond and the other a double bond.

28. (canceled)

29. The method according to claim 27, wherein the RNA virus is HIV-1.

30. The method according to claim 27, wherein R¹ is selected from NR′, O, S, OR′, SR′, SOR′, SO₂R′, NR′₂, and 5- to 10-membered heterocyclyl, wherein each R′ is independently selected from H, C₁₋₆ alkyl, C₃₋₇ cycloalkyl, C₁₋₆ haloalkyl, C₆₋₁₂ aryl, (C₆₋₂₂)aryl(C₂₋₆)alkyl, and 5- to 10-membered heterocyclyl, or two R′ groups form, together with the atom to which they are attached, a 5- to 10-membered heterocyclyl.

31. (canceled)

32. The method according to claim 27, wherein R² is selected from OR′, SR′, SOR′, SO₂R′, C(O)R′, and C(O)OR, wherein R′ is independently selected from C₁₋₆ alkyl, C₃₋₇ cycloalkyl, C₁₋₆ haloalkyl, C₆₋₁₂ aryl, (C₆₋₁₂)aryl(C₁₋₆)alkyl, and 5- to 10-membered heterocyclyl, optionally substituted.

33. (canceled)

34. (canceled)

35. The method according to claim 27, wherein R³, R⁴, R⁵, R⁶, and R⁷ are independently selected from H, halogen, OR′, C₁₋₆ alkyl, and C₁₋₆ haloalkyl, wherein R′ is independently selected from H, C₁₋₆ alkyl, and C₁₋₆ haloalkyl.

36. (canceled)

37. (canceled)

38. (canceled)

39. (canceled)

40. The method according to claim 27, wherein the compound of formula (III) is 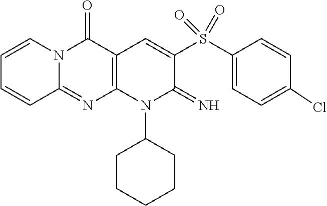 [SMILES: N=c1c(S(=O)(=O)c2ccc(Cl)cc2)cc2c(=O)n3ccccc3nc2n1C1CCCCC1]
or a salt or a solvate thereof.
